(19) United States
(12) Patent Application Publication
(10) Pub. No.: US 2014/0303604 A1
(43) Pub. Date: Oct. 9, 2014

(54) Left Heart Vent Catheter

(71) Applicants: Robert E. Michler, Riverside, CT (US); Albert N. Santilli, Pepper Pike, OH (US)

(72) Inventors: Robert E. Michler, Riverside, CT (US); Albert N. Santilli, Pepper Pike, OH (US)

(21) Appl. No.: 14/216,214

(22) Filed: Mar. 17, 2014

Related U.S. Application Data
(60) Provisional application No. 61/801,957, filed on Mar. 15, 2013.

(51) Int. Cl. A61M 25/10

(52) U.S. Cl. CPC A61M 25/10; A61M 2025/1093
USPC 604/540

(57) ABSTRACT

A left heart vent catheter that includes an elongate tube having a hollow passageway is provided with a plurality of openings and a balloon near the distal end. The balloon can be inflated after the catheter is in place so as to engage a desired part of the heart such as the aortic valve or tricuspid valve and thereby prevent undesired withdrawal of the catheter. The openings near the distal end permit fluid to be withdrawn from the heart through the hollow passageway. After the surgical procedure has been completed, the balloon can be collapsed and the catheter can be withdrawn.

### PRIORITY CLAIM

The present application claims priority from, and incorporates by reference herein for all purposes, provisional application Ser. No. 61/801,957, filed Mar. 15, 2013 by Robert E. Michler and Albert N. Santilli.

### BACKGROUND OF THE INVENTION

1. Field of the Invention

The present invention relates to left heart vent catheters.

2. Description of the Prior Art

In the course of conducting coronary surgery, it sometimes is necessary to provide a direct or indirect vent of the left ventricle. This is accomplished by inserting an elongate, narrow, hollow tube into the ventricle. The distal end of the tube has a plurality of openings that permit air and blood to enter the tube and be conveyed out of the body.

A commercially known left heart vent catheter is marketed by Medtronic, Inc. of Minneapolis, Minn. under the trademark DLP. A specification sheet can be found at http://www.medtronic.com/mics/documents/200805572_EN.pdf (page VII-5). The catheter in question and the specification sheet are incorporated herein by reference. The catheter in question is provided with a so-called guidewire introducer that maintains the catheter in a desired shape as it is being pushed into place. The guidewire introducer is withdrawn after the distal end of the catheter has been properly positioned, leaving the hollow tube in place to receive and discharge air and blood.

A problem with the catheter in question is that it can shift or otherwise move during the course of a surgical procedure. In particular, the catheter can be withdrawn if care is not taken. In order to prevent undesired movement or withdrawal of the catheter, it usually is manually held in place or is sutured in place. Both of these approaches to retaining the catheter in place have a number of drawbacks such as the need to dedicate a person to carrying out the task of taking the time to suture the catheter to an appropriate portion of the patient's body.

### SUMMARY OF THE INVENTION

The present invention provides a new and improved technique for holding left heart vent catheters in place. More specifically, a left heart vent catheter is provided with a balloon near the distal end. The balloon can be inflated after the catheter is in place so as to engage a desired part of the heart such as the aortic valve or tricuspid valve and thereby prevent undesired withdrawal of the catheter. After the surgical procedure has been completed, the balloon can be collapsed and the catheter can be withdrawn.

In the preferred embodiment, a first, elongate tube defines a first passageway. The first tube has a tip end, a central portion, and an exit end, the tip end being provided with openings that establish fluid communication with the first passageway. The tip end and the central portion each have an outer diameter, the outer diameter of the tip end and the central portion being such that they can extend through the patient's inferior or superior vena cava or the right pulmonary vein and the tip end can extend into the patient's left ventricle.

An expansible balloon is disposed adjacent the openings in the tip end and surrounds at least a portion of the first tube. The balloon is positioned intermediate the openings in the tip end and the central portion. The balloon when expanded has an outer diameter, the outer diameter of the expanded balloon being such that it engages the patient's aortic valve or tricuspid valve and prevents retraction of the tip end from the left ventricle.

A second passageway extends along at least a portion of the length of the tube. The second passageway establishes fluid communication with the balloon. A branch tube projects from the side of the first tube in the region of the exit end. The branch tube is in fluid communication with the second passageway such that the balloon can be inflated, typically by injecting saline solution by means of a syringe attached to the branch tube.

The invention eliminates the need to provide a surgical assistant for the purpose of holding the catheter in place, and it avoids the need to take the time and trouble to suture the catheter in place. The invention provides a fast, easily operated, effective way to maintain the desired position of the catheter during the course of a surgical procedure.

### BRIEF DESCRIPTION OF THE DRAWINGS

The foregoing and other features and advantages of the present invention will become apparent to those skilled in the art to which the present invention relates upon reading the following description with reference to the accompanying drawings, in which like reference characters refer to like elements through the different figures and in which:

FIG. 1 is a side elevational view, partly in section, of a left heart vent catheter according to the invention with a guidewire introducer removed;

FIG. 2 is a side elevational view of a guidewire introducer used with the present invention; and

FIG. 3 is a cross-sectional view of the catheter of FIG. 1 taken along a section indicated by line 3-3 in FIG. 1.

### DESCRIPTION OF THE PREFERRED EMBODIMENT

Referring now to the drawings, a left heart vent catheter according to the invention includes an elongate first tube 12 made of latex, vinyl, silicone, or similar material that defines a first, hollow passageway 14. The tube 12 has a distal end portion 16, a central portion 18, and an exit end portion 20. The end of the distal end portion 16 is closed by a rounded bullet tip 21. A plurality of small openings 22 are formed in the tip end 16, adjacent the rounded tip 21. Preferably, four openings 22 are provided, each approximately 2 mm in diameter. The openings 22 are disposed circumferentially about the tube 12 in order to establish fluid communication with the passageway 14 and to maintain structural integrity of the tube 12.

A thin, circumferentially extending, expansible balloon 24 is included as part of the tip end portion 16. The balloon 24 is connected to the tube 12 by means of a second, hollow passageway 26. The balloon 24 is a double lumen balloon that can accommodate at least 12 cc of saline solution.

The second passageway 26 either is included as part of the wall that defines the tube 12 (see FIG. 3) or it can be part of a separate tube that is disposed within the passageway 14. The second passageway 26 terminates in a branch tube 28 that projects from the side of the first tube 12 adjacent the exit end portion 20. A small syringe (about 35 ml) (not shown) is adapted to be connected to the branch tube 28 by means of a luer lock connector. A syringe, suction line, or drain tube (not shown) can be connected to an open end 30 of the exit end portion 20 in order to drain blood, air or other fluid from the passageway 14.

A plurality of small openings 32 are formed in the tip end 16, adjacent the balloon 24, but on the proximal side thereof. Preferably, four openings 22 are provided, each approximately 2 mm in diameter. As with the openings, 22, the openings 32 are disposed circumferentially about the tube 12 in order to establish fluid communication with the passageway 14 and to maintain structural integrity of the tube 12.

A elongate guidewire introducer 34 (FIG. 2) has a large formation 36 at the proximal end and a rounded tip 38 at the distal end. The guidewire introducer can be made of a rigid material but more preferably is made of a malleable material such as metal.

If desired, a number of visual markers 40, 42, 44 can be disposed on the outside of first tube 12. The markers 40, 42, 44 are evenly spaced at 10 cm intervals along the length of the tube 12. The first marker 40 preferably is located 10 cm from the tip 21, the second marker preferably is located 20 cm from the tip 21 and the third marker 44 preferably is located 30 cm from the tip 21. The marker 40 consists of a single line, the marker 42 consists of two spaced lines, and the marker 44 consists of three spaced lines. The 10 cm measurement for the first marker 40 is to the line, the 20 cm measurement for the second marker 40 is to a position exactly between the two spaced lines, and the 30 cm measurement for the third marker 44 is to the middle line. By the use of one, two and three lines, respectively, for the markers 40, 42, and 44, the surgeon will be prompted to know that the markers are located at 10, 20, and 30 cm from the tip 21.

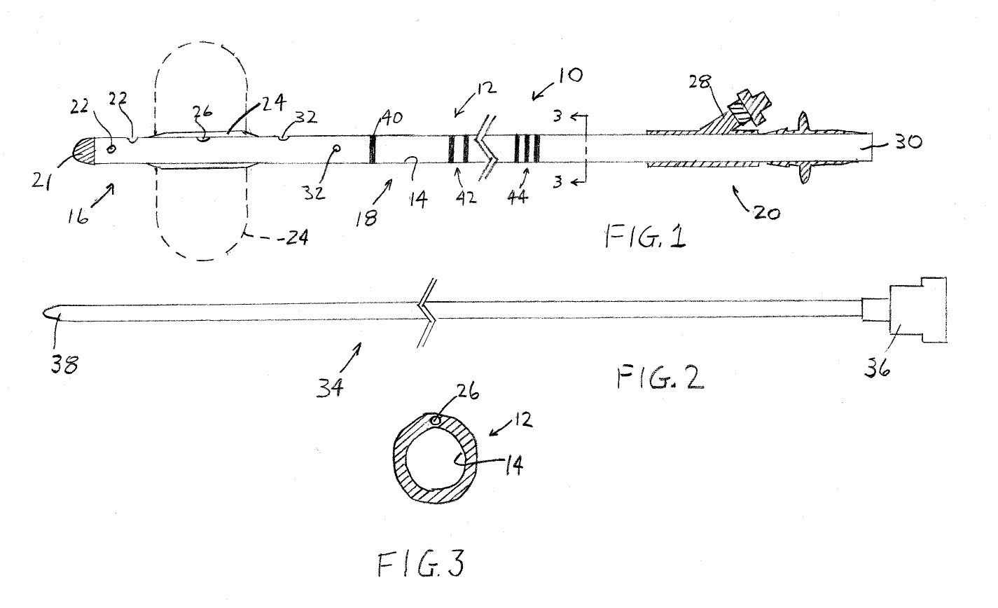

In addition to the dimensions previously given, certain other dimensions are pertinent. Indeed, since the invention is intended for use in coronary surgery, certain of the dimensions are important or critical and not merely approximations. The maximum diameter of the inflated balloon 24, as indicated in FIG. 1 by the dashed lines, should not exceed 3 cm. The distal side of the balloon 24 should be spaced 2.5-3 cm from the end of the rounded tip 21. The maximum width of the balloon should be 1.5 cm. The openings 22 should be disposed between the tip 21 and the distal side of the balloon 24, but should be at least 1 cm away from the distal side of the balloon 24. Similarly, the openings 32 should be disposed close to the balloon 24 on the proximal side thereof, but should be at least 1 cm away from the proximal side of the balloon 24.

The length of the combined distal end portion 16 and the central portion 18 is approximately 15.0 inches, while the exit end portion 20 is approximately 2.2 inches long. The tube 12 has an outer diameter of about 0.213 inch and an inner diameter of about 0.105 inch. The second passageway 26 has a diameter of about 0.030 inch.

### Operation

In use as might typically occur during the course of a surgical procedure, the balloon 24 is collapsed. The rounded tip 38 of the guidewire introducer 34 is inserted into the open end 30 of the hollow passageway 14. The entire guidewire introducer then is inserted into the passageway 14 by grasping and pushing the formation 36. After the guidewire introducer is in place, and if it is made of a malleable material, the tube 12 can be bent into a shape desired by the surgeon. Thereafter, the distal end portion 16 is inserted into the heart through an opening such as the inferior or superior vena cava or the right pulmonary vein. The extent to which the rounded tip 21 is inserted into the heart can be gauged by the surgeon through the use of the markers 40, 42, 44.

After the distal end 16 has been properly positioned, the balloon 24 is inflated with about 5-10 cc of saline solution by a syringe connected to the branch tube 28. The balloon 24 will bear against the aortic valve or the tricuspid valve, thereby preventing the catheter 10 from being withdrawn from the heart during the course of the surgical procedure. While the distal end portion is in the heart, blood, air or other fluid can be drained or withdrawn under vacuum through the openings 22 and the hollow passageway 14. After there is no need for the catheter 10, the balloon 24 can be collapsed so as to permit the catheter 10 to be withdrawn from the heart.

By using the catheter 10 according to the invention, there no longer is a need to provide a surgical assistant for the purpose of holding a catheter in place. The invention also avoids the need to take the time and trouble to suture a catheter in place. The invention provides a fast, easily operated, effective way to maintain the desired position of the catheter 10 during the course of a surgical procedure.

Although the invention has been described in detail with reference to particular examples and embodiments, the examples and embodiments contained herein are merely illustrative and are not an exhaustive list. Variations and modifications of the present invention will readily occur to those skilled in the art. The present invention includes all such modifications and equivalents.

What is claimed is:

1. A left heart vent catheter for venting a patient's left ventricle, comprising:
a first, elongate tube that defines a first passageway, the first tube having a tip end, a central portion, and an exit end, the tip end being provided with openings that establish fluid communication with the first passageway;
the tip end and the central portion each having outer diameter, the outer diameter of the tip end and the central portion being such that they can extend through the patient's inferior or superior vena cava or the right pulmonary vein and the tip end can extend into the patient's left ventricle;
an expansible balloon disposed adjacent the openings in the tip end and surrounding at least a portion of the first tube, the balloon being positioned intermediate the openings in the tip end and the central portion, the balloon when expanded having an outer diameter, the outer diameter of the expanded balloon being such that it engages the patient's aortic valve or tricuspid valve and prevents retraction of the tip end from the left ventricle;
a second passageway extending along at least a portion of the length of the tube, the second passageway establishing fluid communication with the balloon; and
a branch tube projecting from the side of the first tube in the region of the exit end, the branch tube being in fluid communication with the second passageway.

2. The left heart vent catheter of claim 1, wherein the tube includes a wall, and the second passageway is formed as part of the wall.

3. The left heart vent catheter of claim 1, further comprising a second tube that is disposed within the first passageway, the second tube defining the second passageway.

4. The left heart vent catheter of claim 1, wherein the first tube is made of a material selected from the group consisting of latex, vinyl, and silicone.

5. The left heart vent catheter of claim 1, wherein the elongate tube is flexible and further comprising an elongate guidewire introducer of a size and shape to be insertable into and removable from the first passageway.

6. The left heart vent catheter of claim 5, wherein the elongate guidewire introducer is made of a malleable metal.

7. The left heart vent catheter of claim 1, wherein the openings in the tip end are 2 mm in diameter.

8. The left heart vent catheter of claim 1, wherein the balloon when inflated has a maximum width of 1.5 cm and a maximum diameter of 3 cm.

9. The left heart vent catheter of claim 1, wherein the openings in the tip end are on a distal side of the balloon and further comprising openings in the tip end disposed on a proximal side of the balloon.

10. The left heart vent catheter of claim 9, wherein the openings in the tip end on the distal side of the balloon and the openings in the tip end on the proximal side of the balloon are located at least 1 cm from the balloon.

11. A method of venting the left ventricle of a patient's heart, comprising the steps of:
providing a left heart vent catheter having:
an elongate tube made of a flexible material that defines a first passageway, the elongate tube having a tip end, a central portion, and an exit end, the tip end being provided with openings that establish fluid communication with the first passageway;
an expansible balloon disposed adjacent the openings in the tip end and surrounding at least a portion of the elongate tube, the balloon being positioned intermediate the openings in the tip end and the central portion;
a second passageway extending along at least a portion of the length of the elongate tube, the second passageway establishing fluid communication with the balloon; and
a branch tube projecting from the side of the elongate tube in the region of the exit end, the branch tube being in fluid communication with the second passageway;
inserting the tip end and the balloon into the patient's left ventricle by passing the elongate tube through the patient's inferior or superior vena cava or the right pulmonary vein;
inflating the balloon by pumping fluid into the second passageway through the branch tube until the balloon reaches a size to engage either the aortic valve or the tricuspid valve; and
draining fluid within the patient's left ventricle through the openings in the tip end and the first passageway.

12. The method of claim 10, wherein the fluid pumped into the second passageway is saline solution.

13. The method of claim 10, further comprising the steps of:
providing a guidewire introducer; and
placing the guidewire introducer in the first passageway, the steps of providing a guidewire introducer and placing the guidewire introducer in the first passageway being conducted prior to the step of inserting the tip end and the balloon into the patient's left ventricle by passing the elongate tube through the patient's inferior or superior vena cava or the right pulmonary vein.

14. The method of claim 13, further comprising the step of removing the guidewire introducer from the first passageway after the tip end and the balloon have been inserted in the patient's left ventricle.
